(19) United States
(12) Patent Application Publication
(10) Pub. No.: US 2017/0151414 A1
(43) Pub. Date: Jun. 1, 2017

(54) CATHETER FOR THE DELIVERY OF THERAPEUTIC FLUID

(71) Applicant: COMBAT MEDICAL SL, Ajalvir Madrid (ES)

(72) Inventor: Alberto Martinez Albalat, Ajalvir Madrid (ES)

(73) Assignee: COMBAT MEDICAL SL, Ajalvir Madrid (ES)

(21) Appl. No.: 15/322,867

(22) PCT Filed: Jul. 1, 2015
(86) PCT No.: PCT/EP2015/064959

§ 371 Date: Dec. 29, 2016

(30) Foreign Application Priority Data
Jul. 18, 2014 (GB) 1412842.5

(51) Int. Cl. A61M 25/00; A61F 7/12

(52) U.S. Cl. CPC A61M 25/0026; A61F 7/12; A61F 2007/0059

(57) ABSTRACT

A catheter for the delivery of a therapeutic fluid to a patient's cavity is provided. The catheter includes an inflow lumen for delivering fluid to the patient's cavity; an outflow lumen for recovering fluid from the patient's cavity; an electrode located, in use, in the patient's cavity; and structure for supplying power to the electrode. Also provided is a system for the delivery of a therapeutic fluid to a patient's cavity, including the catheter, and a method for delivering a therapeutic fluid into a patient's cavity, including the step of using the catheter.

The present invention relates to a catheter device and corresponding system and method for the improved administration of a therapeutic fluid to a patient. More particularly, the present invention is based on the delivery and/or recirculation of heated ionised drug solutions to specific target areas using a low-voltage electrical current.

Chemotherapy uses cytotoxic drugs to destroy cancer cells. Traditionally, the cytotoxic drugs are injected directly into a patient's bloodstream or are provided in the form of tablets or capsules that breakdown such that the cytotoxic drugs enter the patient's bloodstream indirectly. Such techniques rely on the cytotoxic drugs circulating within the patient's bloodstream to reach the cancer cells.

Administration methods by-passing the patient's bloodstream have been developed, such as intra-vesical chemotherapy (IVC) for the treatment of bladder cancer and intra-peritoneal chemotherapy (IPC) for the treatment of ovarian cancer. In WC and IPC treatments, the cytotoxic drugs are delivered to a patient's bladder or abdomen in the form of fluids via a catheter. In intra-vesical chemotherapy, the catheter is inserted via a patient's urethra, whereas in cytoreductive surgery the catheter is inserted via a hole cut in the wall of a patient's abdomen. The fluids may be added to a patient's bladder or abdomen using a first catheter allowed to circulate within the bladder or abdomen and then withdrawn from the bladder or abdomen using a second catheter. These methods deliver the cytotoxic drugs directly to the cancer cells with minimal absorption of the drug into the bloodstream and hence with fewer side-effects than traditional chemotherapy.

More recently, techniques such as Chemo-Hyperthermia Therapy (CHT) and Electromotive Drug Administration (EMDA) have been introduced.

Chemo-hyperthermia therapy is a variation of treatments such as IVC and IPC in which the cytotoxic drugs which is circulated within the bladder or abdomen is heated to a few degrees above body temperature to make the drugs more effective in killing the cancer cells. Hyperthermia has been shown to alter intracellular distribution of drugs, while increasing both their metabolism and reaction rates. In particular, hyperthermia has been shown to increase drug uptake by neoplastic cells while at the same time inhibiting DNA repair in damaged neoplastic cells. An application of this method is described in further detail in the Inventor's own EP2654861.

An alternative drug administration method is electromotive drug administration, which involves three mechanisms, iontophoresis, electrophoresis and electroporation, all of which are based on the application of an electrical current to migrate charged particles. Each of these mechanisms is known in the art and described, for example, in U.S. Pat. No. 5,779,661. In practice, a first electrode connected to a source of electric current is inserted inside the patient and a second electrode is applied externally. An ionised drug solution is delivered adjacent to the target area and an electrical current is applied to facilitate the migration of the drug(s) into the tissues to be treated.

Both methods have shown promising results. However, these treatments involve the administration of the drug(s) in multiple sessions, typically 6 to 12 sessions a year and during each session a catheter or other device must be inserted into the patient. This is clearly an exhausting procedure, especially as most patients would have undergone surgery beforehand and would be in a recovery period. In addition, there is still a significant risk of reappearance of cancerous cells despite treatments. There is therefore a need for a treatment to improve patient comfort and to lower the recurrence rate of tumours.

It is an object of the invention to at least alleviate the above-mentioned disadvantages, or to provide an alternative to existing products.

According to a first aspect of the invention, there is provided a catheter for the delivery of a therapeutic fluid to a patient's cavity. The catheter comprises an inflow lumen for delivering fluid to the patient's cavity; an outflow lumen for recovering fluid from the patient's cavity; an electrode which in use is located in the patient's cavity; and means for supplying power to the electrode.

Thus, the catheter according to the present invention enables the delivery, recovery and/or recirculation of a therapeutic fluid and the electromotive administration of the same therapeutic fluid. This device is particularly advantageous when the fluid is heated prior to delivery to combine the beneficial effects of EMDA and chemo-hyperthermia therapy. One would not have previously considered combining the treatments, as they would be applied separately, in two sets of chemotherapy sessions, i.e. 12 to 24 sessions per year. This would be not only extremely exhausting for the patient, but would also increase the risk of tissue trauma and infection due to repetitive catheter insertions. Furthermore, the two sets of chemotherapy have cumulative side effects and could potentially reach toxic drug levels. Finally, there would be an economic impact because of the two sets of medical expenses. Alternatively, if the EMDA and CHT sessions are carried out in turn so as to limit the total number of sessions to 6 to 12 per year, the beneficial effect of each separate therapy is reduced.

The inflow lumen allows the delivery of the therapeutic fluid into the patient's cavity; the outflow lumen allows the recovery of the therapeutic fluid from the patient's cavity. This double-lumen configuration presents many advantages when the catheter is integrated in a therapeutic fluid recirculation system. For example, the heated therapeutic fluid can be delivered without losing heat. Indeed, in a single lumen catheter in which the delivered and recovered fluids circulate in a single channel, the temperature of the delivery fluid will decrease due to mixing with the cooler recovered fluid. In addition, in a single-lumen system, the outgoing fluid will disrupt the ingoing fluid, and vice versa, so that efficient recirculation cannot be achieved. This is not an issue when at least two lumens are provided. Moreover, the double-lumen catheter is advantageous in that the recovered fluid can be treated, for example filtered, before being recirculated so as to minimise or prevent the risk of blockage in the system.

Preferably, the catheter further comprises one or more inflow ports and one or more outflow ports, said inflow and outflow ports being located, in use, in the patient's cavity. The inflow and outflow ports are preferably located adjacent the distal tip of the catheter. Furthermore, to ensure optimum ionisation, the inflow port is located adjacent the electrode. For optimum spread, the inflow port and the outflow port are positioned at substantially diametrically opposed position from each other. The number, shape and dimensions of the inflow and outflow ports will depend on parameters such as the cavity to be treated, the volume of the fluid to be delivered, the desired flow rate and the like.

Preferably, the inflow lumen and the outflow lumen are integrally formed in the catheter. For example, the catheter and the lumens may be integrally moulded from the same material or the lumen are integrally formed within the material of the catheter. The material of the catheter and/or the lumens is preferably biocompatible and/or non-conductive. Examples of preferred materials for the catheter include, but are not limited to polyvinylchloride (PVC), polyurethane (PU), polyethylene (PE), polypropylene (PP) and silicon-based materials.

The power supply means allows current to be provided from a power source to the catheter electrode. Preferably, the power supply means comprises a power supply cable. In a preferred embodiment, the power supply cable is flexible enough so as to prevent discomfort and injury to the patient and rigid enough to facilitate guiding of the catheter into the cavity. In this embodiment, the material of the catheter is also preferably flexible.

According to a second aspect of the invention, there is provided a system for the delivery of a therapeutic fluid to a patient's cavity. By including a catheter as described above, the system is versatile and can be used to deliver a therapeutic fluid, recover the fluid, recirculate the fluid, heat the fluid, apply EMDA, as individual functions or in combination. As explained above, the combination of these functions can be carried out in a single therapy session with only one catheter insertion.

Preferably, the system further comprises an external electrode located, in use, on an area of the patient's skin. This external electrode may be presented in the form of a pad for ease of application. Preferably, the system further comprises an electrical power source. The external electrode and electrical power source form the electrical circuit including the internal electrode and the power supply means in the catheter.

Preferably, the system further comprises a source of therapeutic fluid comprising at least one or more drug compounds in a solution. For efficient electromotive administration, the drug(s) itself may be ionised and/or be present in an ionised medium. Preferred drugs include but are not limited to chemotherapeutic drugs (e.g. mitomycin) and antibiotics. Ionised media which will not inhibit or decrease the beneficial activity of the drug(s) will be chosen, such as distilled water based media. The therapeutic fluid preferably comprises a liquid or is a liquid.

Preferably, the system further comprises means for selectively excluding or including the source of therapeutic fluid from or in the recirculation system. The means may comprise a valve which can be switched between a first position in which the fluid source is included in the system and a second position in which the fluid source is excluded from the system. The source may be included in the system at the beginning of the procedure in order to introduce the therapeutic fluid into the system. Once the required amount of fluid has been introduced into the system, the fluid source is excluded from the system. This provides an effective way of accurately controlling the amount of fluid being delivered to the patient and recirculated and is particularly advantageous where only small amounts of fluids can be used, for example in IVC procedures.

Preferably, the system further comprises a heating element for heating the therapeutic fluid. In a preferred embodiment, the heating element warms a heat exchanger through which the therapeutic fluid circulates. For example, the therapeutic fluid circulates through a channel in a warming cassette which is positioned in a heating element. A preferred heat exchanger developed by the inventor is described in WO 2012/168451 and comprises a thermo-conductive sheet comprising an outer layer of metal foil and an inner layer of a biocompatible material.

The system according to the present invention can be used as an open system in which the therapeutic fluid is delivered to the patient's cavity, recovered from the cavity, then discarded. More advantageously, the system is used as a closed system is which the fluid recovered from the cavity is recirculated into the patient, thereby enabling an accurate control of the amount of fluid (and therefore drugs delivered), an accurate control of the temperature and pressure of the fluid in the system.

According to a third aspect of the invention, there is provided a method for delivering a therapeutic fluid into a patient's cavity, including a catheter or system as described above. In a preferred embodiment, the method comprises the steps of (a) inserting the catheter into the patient's cavity; (b) delivering the therapeutic fluid through the inflow lumen and recovering the therapeutic fluid through the outflow lumen; and (c) applying a current to the electrode.

Preferably, the method includes the step (d) of recirculating the recovered therapeutic fluid into the inflow lumen.

The method according to the present invention may be an intra-vesical treatment, an intra-peritoneal treatment or an intra-vascular treatment. The expression “body cavity” excludes cavities such as ear cavity, lungs or mouth or other cavities for which this method would not be suitable.

As mentioned above, the various functions of the catheter can be used separately or in combination. In a preferred embodiment, all functions are used steps (b) and (c) are carried out simultaneously so that the patient can benefit from the combined therapeutic effects of EMDA and chemo-hyperthermia, Not only the present method decreases the number of sessions required in a year, but it will be shown hereinafter that a synergic effect can be observed when both techniques are applied simultaneously.

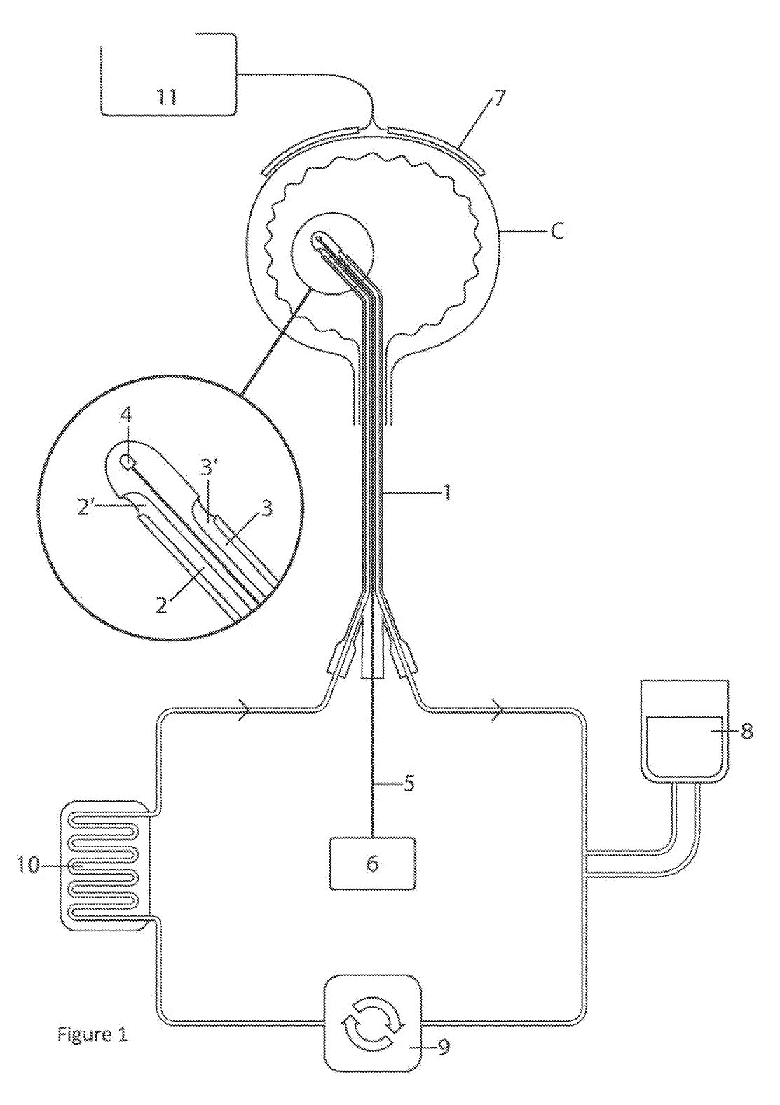

The invention will be further described with reference to FIG. 1 which is a schematic representation of a system comprising a catheter according to the present invention.

Referring to FIG. 1, there is illustrated a catheter 1 for the delivery of a therapeutic fluid to a patient's cavity C, said catheter I comprising an inflow lumen 2 for delivering fluid to the patient's cavity C; an outflow lumen 3 for recovering fluid from the patient's cavity C; an electrode 4 located, in use, in the patient's cavity C; and means 5 for supplying power to the electrode 4.

In this embodiment, the power supply means 5 is a power cable. The catheter I is made of a flexible biocompatible material and the power cable 5 is a flexible cable or wire for ease of manipulation and insertion and minimal discomfort to the patient. The power supply means 5 is enclosed in a non-conductive sheath (not shown). The sheath can be integrally formed in the catheter 1 or the catheter 1 itself can be made of a non-conductive material.

The catheter 1 comprises a generally elongate member within which the inflow lumen 2, outflow lumen 3 and power supply means 5 are enclosed. Various constructions can be considered such as those shown in FIGS. 2A to 2C. The lumens 2, 3 and/or power cable sheath where applicable can be formed as separate channels or be integrally formed in the catheter 1. Other elements can be added to the catheter 1, including sensors (not shown) for measuring the temperature and/or pressure of the fluid entering and/or leaving the cavity.

Within the context of this invention, we will refer to the end of the catheter closest to medical professional as the proximal end of the catheter and to the end of the catheter closest to the patient's cavity as the distal end of the catheter. All the value ranges provided are inclusive of the outermost values.

The distal end of the catheter 1 is bent in order to assist insertion and to minimise tissue trauma. The overall diameter and the diameters of the lumens depend on the cavity to be treated (e.g. smaller for IVC than for IPC), the required flow rate and volume of therapeutic fluid to be circulated. The catheter 1 is made of a biocompatible and non-conductive material.

In FIG. 1, the electrode 4 (sometimes referred to as the “antenna” or “active electrode”) is located at the distal extremity of the catheter and connected to an external electrical power supply 6 through power cable 5. A second electrode 7, which in this instance is a pad, is placed on the patient's skin effectively to close the electrical circuit. The electrode 7 is connected to an electro-motive system 11.

The catheter 1 further comprises an inflow port 2′ in fluid communication with the inflow lumen 2 and the cavity C; and an outflow port 3′ in fluid communication with the outflow lumen 3 and the cavity C. In use (i.e. when the catheter 1 is correctly positioned in the patient's cavity), the inflow and outflow ports 2′ and 3′ are located at the distal end of the catheter and in the patient's cavity so that the therapeutic fluid can be delivered to and recovered from the cavity C. In FIG. 1, the inflow port 2′ is located adjacent the internal electrode 4 for optimum EMDA efficiency.

FIG. 1 also shows a delivery and/or recirculation system comprising a reservoir 8 for the therapeutic fluid, a pumping element 9 for circulating the fluid through the system and a heating element 10 for heating the fluid.

The fluid reservoir 8 contains the therapeutic fluid and can be a syringe, a fluid bag or other types of reservoir. its capacity can be from 50 to 500 ml for IVC procedures and from 50 to 5000 ml. for IPC procedures. This is particularly relevant where a fixed amount of drug(s) and therefore a fixed volume of fluid is required. The system comprises a switch to exclude the reservoir 8 from the system, once the fluid has been delivered into the system. The fluid reservoir 8 can be connected directly to the heating element 10 or to a tubing in the system.

The heating element 10 in this preferred embodiment comprises a fluid heating container through which the fluid circulates and which can be placed in a warming unit, typically between two heating plates. The heating element is adapted to adjust the temperature with a variation of at most +/−1° C. and the temperature of the therapeutic fluid as it enters the patient's cavity ranges from 40 to 44° C. Any numerical value ranges described within the context of the invention include the lowest and highest values.

The present invention is particularly advantageous when the therapeutic fluid is recirculated, i.e. when the fluid recovered from the patient's cavity is recirculated into the cavity. The therapeutic fluid is heated up to the required temperature (the most preferred. temperature being 43° C.). The fluid is then delivered without any heat into the body cavity and maintained at that same temperature because the fluid is being constantly recirculated. The constant vasodilatation thus achieved enables the drug(s) to work more efficiently.

For optimum heating, the pumping element 9, in this instance a peristaltic pump, is positioned before the heating element 10 so that the therapeutic fluid is fed into the heating element 9 by positive pressure. In intra-vesical chemotherapy the pump will typically maintain a recirculation flow of between 1 ml/min to 500 ml/min, but optimum results are obtained at a recirculation flow of between 100 to 350 ml/min.

Temperature and/or pressure sensors can be included in the system to measure the temperature and/or pressure as the fluid enters the patient's cavity, as the fluid exits the patient cavity, before entering the heating element 10 (to protect the heating element).The sensors can provide a direct measure of the fluid's temperature and/or pressure (in-line sensors), be located outside the tubing, outside the patient or inside the patient.

The system can in an “open system” in which the fluid is delivered to the patient's cavity and subsequently removed. In this embodiment, the fluid removed from the patient's cavity is recirculated through the system for redelivery to the cavity. The present system allows the fluid to be delivered at accurate and constant temperature and pressure.

Typically, the system will comprise an integrated control unit that controls the heating element, the pump and/or the electrical power source. Preferably the integrated control unit will contain a control panel and a screen, which screen may display real-time readings. The integrated control unit may also enable the recording of data and may also enable the programming of the system (e.g. a user may choose a program with a specific length of and temperature profile for treatment of a patient).

In use, a catheter 1 fitted to the patient. In an WC procedure, the catheter is inserted via a patient's urethra; in an IPC procedure, the catheter is inserted via an incision in the wall of a patient's abdomen. The catheter 1 is fitted so that the electrode 4, the inflow port 2′ and the outflow port 3′ are located in the patient's cavity, adjacent the area to be treated. A second electrode is fitted to the patient, generally as a pad on the patient's skin.

The therapeutic fluid from a fluid reservoir 8 is injected into the system either through a tubing before the pump 9 and heating element 10, or can be injected into the system though the heating element 10. Once the required amount of therapeutic fluid has been injected, a valve is switched which excludes the reservoir 8 from the system.

The fluid is circulated through the system by the pump 9 and flows through the heating element 10. The circulation pressure can be measured and adjusted prior to entry into the heating element 10. The fluid circulates through a fluid heating container located between two heating plates of a warming unit. The temperature of the fluid can be measured and adjusted in the heating element 10 and/or as it exits the heating element 10. The fluid is then circulated to the inflow lumen 2 of the catheter I and exits into the patient's cavity through inflow port 2′.

In the meantime, a low voltage current is supplied to the internal electrode 4 by the electrical power source 6 through the power cable 3. The recommended voltage ranges from 1 mA to 5 mA. The electric current is applied to the fluid as it exits the catheter and the drug migrates to the targeted areas by iontophoresis and electrophoresis. This migration is further improved by the hyperthermic state created by the heated fluid. The fluid is recovered through the outflow port 3′ into outflow port 3, to be discarded or preferably recirculated in the system.

Thus, from the description above, it can be seen that the present invention provides a good alternative to existing cancer treatment methods and can be used before, during and/or after surgery, as a chemotherapy treatment, to prevent the reoccurrence of cancer and/or for maintenance therapy for high risk patients. In particular, the catheter and system according to the present invention enables the administration of drugs by combining EMDA, chemo-hyperthermia and recirculation. This can be achieved without increasing the number of chemotherapy sessions and associated disadvantages. Furthermore, a synergic effect is obtained by combining the EMDA technology with hyperthermia and lower recurrence rates can be observed.

1. A catheter for the delivery of a therapeutic fluid to a patient's cavity, said catheter comprising an inflow lumen for delivering fluid to the patient's cavity; an outflow lumen for recovering fluid from the patient's cavity; an electrode located, in use, in the patient's cavity; and
means for supplying power to the electrode.

2. The catheter according to claim 1, wherein the electrode is positioned adjacent the distal tip of the catheter.

3. The catheter according to claim 1, wherein the means for supplying power comprises a power supply cable.

4. The catheter according to claim 1, wherein the inflow lumen and the outflow lumen are integrally formed within the catheter.

5. The catheter according to claim 1, further comprising one or more inflow ports in fluid connection with the inflow lumen and one or more outflow ports in fluid connection with the outflow lumen, wherein the inflow and outflow ports are located, in use, in the patient's cavity.

6. The catheter according to claim 5, wherein the one or more inflow ports and the one or more outflow ports are positioned adjacent the distal tip of the catheter.

7. The catheter according to claim 5, wherein the inflow port(s) are positioned adjacent the electrode.

8. The catheter according to claim 1, wherein the catheter is flexible.

9. A system for the delivery of a therapeutic fluid to a patient's cavity, said system comprising a catheter according to claim 1.

10. The system according to claim 9, further comprising an external electrode located, in use, on the patient's skin.

11. The system according to claim 9, further comprising an electrical power source to supply current to the electrode(s).

12. The system according to claim 9. further comprising a source of therapeutic fluid, wherein said fluid comprises an ionised solution comprising one or more drug compounds.

13. The system according to any one of claim 9, further comprising a source of therapeutic fluid, wherein said fluid comprises a solution comprising one or more drug compounds in ionised form or capable of being ionised.

14. The system according to claim 9, further comprising a heating element for heating the therapeutic fluid.

15. The system according to claim 9, wherein the system is a fluid recirculation system.

16. The system according to any one of claim 15, further comprising means for selectively excluding or including the source of therapeutic fluid from or in the recirculation system.

17. A method for delivering a therapeutic fluid into a patient's cavity, comprising the step of using a catheter according to claim 1.

18. The method according to claim 17 comprising the steps of:
a) inserting the catheter into the patient's cavity;
b) delivering the therapeutic fluid through the inflow lumen and recovering the therapeutic fluid through the outflow lumen: and
c) applying a current to the electrode.

19. The method according to claim 18, wherein steps (b) and (c) are carried out simultaneously.

20. The method according to claim 17, wherein the method is an intra-vesical treatment.

21. The method according to claim 17, wherein the method is an intra-peritoneal treatment.

22. (canceled)

23. (canceled)

24. (canceled)
